(19) United States
(12) Patent Application Publication
(10) Pub. No.: US 2025/0340628 A1
(43) Pub. Date: Nov. 6, 2025

(54) COMPOSITIONS AND METHODS FOR TREATING MACROPHAGE ACTIVATION SYNDROME

(71) Applicant: TJ Biopharma (Hangzhou) Co., Ltd., Hangzhou, Zhejiang (CN)

(72) Inventors: Jian DING, Hangzhou (CN); Yajuan WANG, Hangzhou (CN); Su ZHANG, Hangzhou (CN); Wenyu GUO, Hangzhou (CN); Jingwu ZANG, Shanghai (CN)

(21) Appl. No.: 19/144,648

(22) PCT Filed: Dec. 28, 2023
(86) PCT No.: PCT/CN2023/142663

§ 371 Date: Jun. 30, 2025

(30) Foreign Application Priority Data
Dec. 30, 2022 (WO) PCT/CN2022/144127

(51) Int. Cl. C07K 16/24; A61K 31/573; A61K 31/58; A61K 39/00; A61P 37/06

(52) U.S. Cl. CPC C07K 16/243; A61K 31/573; A61K 31/58; A61P 37/06; A61K 2039/505; A61K 2039/545; C07K 2317/76

(57) ABSTRACT

The present disclosure provides compositions and methods for treating and preventing macrophage activation syndrome (MAS) or hemophagocytic lymphohistiocytosis (HLH). The method entails administering to the patient in need thereof an antibody or fragment thereof having specificity to a human GM-CSF protein.

### BACKGROUND

Haemophagocytic lymphohistiocytosis (HLH) is a fatal autosomal recessive disease that manifests in early childhood. This disease is characterized by fever, hepatosplenomegaly, cytopenia and widespread infiltration of vital organs by activated lymphocytes and macrophages. Macrophage activation syndrome (MAS), also known as secondary HLH, is a rare and serious, life-threatening disease. MAS is considered a hyperinflammatory ‘cytokine storm’ state associated with rheumatic disease or other triggers. The precise mechanisms of MAS are poorly understood. However, regardless of the initiating event for these diseases, the final condition is defined by an overactive immunity that leads to chronic inflammation and hemophagocytosis. MAS is characterized with fever, hyperferritinaemia, haemophagocytosis, cytopenias (including pancytopenia), and hepatosplenomegaly.

Existing treatments for HLH/MAS rely on various immune suppressive regimens of dexamethasone or other steroids, cyclosporine, and etoposide. These therapies are, themselves, toxic and add to the mortality of the already sick patient. Therefore, newer and safer therapies are highly needed in patients with HLH/MAS.

### SUMMARY

The present disclosure provides compositions and methods for treating and preventing haemophagocytic lymphohistiocytosis (HLH) or macrophage activation syndrome (MAS). The method entails administering to the patient in need thereof an antibody or fragment thereof having specificity to a human GM-CSF protein, such as IMH001. The treatment using the anti-GM-CSF antibodies provided herein showed potent efficacy to delay disease progression.

One embodiment of the present disclosure provides a method for treating or preventing macrophage activation syndrome (MAS) or hemophagocytic lymphohistiocytosis (HLH) in a patient in need thereof, comprising administering to the patient an antibody or fragment thereof having specificity to a human GM-CSF protein.

One embodiment of the present disclosure provides an antibody or fragment thereof having specificity to a human GM-CSF protein for use in treating or preventing macrophage activation syndrome (MAS) or hemophagocytic lymphohistiocytosis (HLH) in a patient in need thereof.

One embodiment of the present disclosure provides use of an antibody or fragment thereof having specificity to a human GM-CSF protein in the manufacture of a medicament for treating or preventing macrophage activation syndrome (MAS) or hemophagocytic lymphohistiocytosis (HLH) in a patient in need thereof.

In some embodiments, the antibody comprises a heavy chain variable region (VH) comprising the amino acid sequence of SEQ ID NO:1 and a light chain variable region (VL) comprising the amino sequence of SEQ ID NO:2. In some embodiments, the antibody comprises a human IgG1 Fe.

In some embodiments, the patient has a disease or condition selected from the group consisting of systemic-onset juvenile idiopathic arthritis (SoJIA), systemic lupus erythematosus (SLE), Kawasaki disease, and adult-onset Still's disease.

In some embodiments, the patient has a blood ferritin level that is at least 300 ng/ml, or at least 350 ng/ml, 400 ng/ml, 450 ng/ml, 500 ng/ml, 550 ng/ml, 600 ng/ml, 650 ng/ml, 684 ng/ml, 700 ng/ml, 720 ng/ml, 740 ng/ml, 750 ng/ml, 760 ng/ml, 784 ng/ml, 800 ng/ml, 820 ng/ml, 840 ng/ml, 850 ng/ml, 860 ng/ml, 884 ng/ml, 900 ng/ml, 950 ng/ml, or 1000 ng/ml.

In some embodiments, the patient has a hemoglobin level that is lower than 11 g/dL, or lower than 10.5 g/dL, 10 g/dL, 9.5 g/dL, 9 g/dL, 8.5 g/dL, 8 g/dL, 7.5 g/dL, or 7 g/dL.

In some embodiments, the patient has a platelet count that is lower than 140×10⁹/L, or lower than 130×10⁹/L, 120×10⁹/L, 110×10⁹/L, 100×10⁹/L, 90×10⁹/L, 80×10⁹/L, 70×10⁹/L, or 60×10⁹/L.

In some embodiments, the patient has a neutrophil count that is lower than 1.4×10⁹/L, or lower than 1.3×10⁹/L, 1.2×10⁹/L, 1.1×10⁹/L, 1.0×10⁹/L, 0.9×10⁹/L, 0.8×10⁹/L, 0.7×10⁹/L, or 0.6×10⁹/L.

In some embodiments, the patient has a fasting triglyceride level that is higher than 2.49 mmol/L, or higher than 2.55 mmol/L, 2.6 mmol/L, 2.66 mmol/L, 2.72 mmol/L, 2.77 mmol/L, 2.83 mmol/L, 2.89 mmol/L, 2.94 mmol/L, 3 mmol/L, 3.06 mmol/L, 3.11 mmol/L, 3.17 mmol/L, 3.23 mmol/L, 3.28 mmol/L, 3.34 mmol/L, or 3.4 mmol/L.

In some embodiments, the patient has a blood fibrinogen level that is lower than 1.5 g/L, or lower than 1.4 g/L, 1.3 g/L, 1.2 g/L, 1.1 g/L, 1.0 g/L, 0.9 g/L, 0.8 g/L, 0.7 g/L, 0.6 g/L, or 0.5 g/L.

In some embodiments, the patient has an elevated soluble CD163 level as compared to a healthy individual. In some embodiments, the patient has an elevated soluble IL-2 receptor level as compared to a healthy individual.

In some embodiments, the antibody or antigen binding fragment thereof is administered at 0.3 mg/kg to 10 mg/kg. In some embodiments, the antibody or antigen binding fragment thereof is administered once a week, once every two weeks, or once a month. In some embodiments, the antibody or antigen binding fragment thereof is administered intravenously or subcutaneously.

In some embodiments, the antibody is provided in a formulation comprising: 20 mg/ml-200 mg/ml of the antibody; 10 mM-30 mM of a buffering agent; 130 mM-250 mM of an isotonicity modifier; and 0.01% (w/v)-0.03% (w/v) of a surfactant, wherein the formulation has a pH of 4.5-7.5.

In some embodiments, the formulation comprises 50-150 mg/ml of the antibody, 10-20 mM histidine, 200-220 mM sucrose, and 0.01-0.03% (w/v) polysorbate 80, at a pH of about 5.5-6.1.

In some embodiments, the formulation comprises about 100 mg/ml of the antibody, about 20 mM histidine, about 220 mM sucrose, and about 0.02% (w/v) polysorbate 80, at a pH of about 5.8.

In some embodiments, the method further comprises administering to the patient a glucocorticoid, cyclosporine or anakinra. In some embodiments, the glucocorticoid is selected from the group consisting of cortisol, cortisone, prednisone, prednisolone, methylprednisolone, dexamethasone, betamethasone, triamcinolone, deflazacort, fludrocortisone acetate, deoxycorticosterone acetate, aldosterone, and beclometasone.

### BRIEF DESCRIPTION OF THE DRAWINGS

FIG. 1 shows the body weight changes in the treatment groups.

FIG. 2 shows the RBC (panel A), HGB (panel B), NEUT % (panel C), MONO % (panel D) changes in the treatment groups.

FIG. 3 shows the total bone marrow viable cells changes in the treatment groups.

FIG. 4 shows fibrinogen (panel A), triglycerides (panel B) and ferritin (panel C) levels in the treatment groups.

FIG. 5 shows the histopathology evaluation of hemophagocytosis (panel A), inflammatory infiltration (panel B) and lymphocyte exhaustion (panel C) in the treatment groups.

### DETAILED DESCRIPTION

Before describing the disclosure in detail, it is to be understood that this disclosure is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se. In case of doubt, or should there be no art recognized common understanding regarding the error range for a certain value or parameter, “about” means±1%, 2%, 3%, 4%, 5%, 6%, 7%, 8%, 9%, or 10% of this value or parameter.

It is understood that aspects and embodiments of the disclosure described herein include “comprising,” “consisting,” and “consisting essentially of aspects and embodiments.

The term “antibody”, as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody”. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Unless specifically indicated otherwise, the term “antibody”, as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody, such as F (ab′) 2, F (ab) 2, Fab′, Fab, Fv, scFv and the like, that retain the ability to specifically bind to human GM-CSF or an epitope thereof.

The term “binds specifically”, or the like, means that an antibody or antigen-binding fragment thereof forms a complex with an antigen that is relatively stable under physiologic conditions. Specific binding can be characterized by a dissociation constant of at least about 1×10⁻⁸ sM or greater. Methods for determining whether two molecules specifically bind are well known in the art and include, for example, equilibrium dialysis, surface plasmon resonance, and the like. An isolated antibody that specifically binds human GM-CSF may, however, have cross-reactivity to other antigens, such as GM-CSF molecules from other species (orthologs). In the context of the present disclosure, multispecific (e.g., bispecific) antibodies that bind to human GM-CSF as well as one or more additional antigens are deemed to “specifically bind” human GM-CSF. Moreover, an isolated antibody may be substantially free of other cellular material or chemicals.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “prevention” or “prophylaxis” as used herein means the prevention of or protective treatment for a disease or disease state. Prevention of a disease or disease state can include reduction (e.g., mitigation) of one or more symptoms of the disease or disease state, e.g., relative to a reference level (e.g., the symptom(s) in a similar subject not administered the treatment). Prevention can also include delaying onset of one or more symptoms of the disease or disease state, e.g., relative to a reference level (e.g., the onset of the symptom(s) in a similar subject not administered the treatment). In embodiments, a disease is a disease described herein.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

### Treatment of Macrophage Activation Syndrome (MAS) or Hemophagocytic Lymphohistiocytosis (HLH)

The instant inventors tested the anti-GM-CSF antibody, IMH001, in an animal model for the macrophage activation syndrome (MAS) or hemophagocytic lymphohistiocytosis (HLH). The animals in the MAS were immunodeficient and expressed human GM-CSF and IL-3 cytokines. They showed progressive disease with many of the hallmarks of MAS and rapidly developed a progressive anemia and weight loss.

When treated with IMH001, as compared to controls, the animals showed reduced weight loss (or even weight gain), ameliorated anemia (improved red blood cell counts, hemoglobin and hematocrit), and inhibited ferritin increase. Such in vivo data, therefore, demonstrated the efficacy of IMH001 in treating MAS or HLH.

In accordance with one embodiment of the present technology, therefore, provided is a method for treating or preventing macrophage activation syndrome (MAS) or hemophagocytic lymphohistiocytosis (HLH). In some embodiments, the method entails administering to a patient in need an anti-GM-CSF antibody or antigen-binding fragment. As used herein, the term “GM-CSF” means human granulocyte macrophage colony-stimulating factor. GM-CSF, also known as colony stimulating factor 2 (CSF2), is a monomeric glycoprotein secreted by macrophages, T cells, mast cells, NK cells, endothelial cells and fibroblasts that functions as a cytokine. The pharmaceutical analogs of naturally occurring GM-CSF are also referred to as sargramostim and molgramostim. Antibodies to human GM-CSF are described in, for example, WO2006122797, WO2015028657, and WO2018050111.

In some embodiments, the anti-GM-CSF antibody is IMH001, which is an humanized IgG1 antibody that includes a heavy chain variable region (VH) of SEQ ID NO:1 and a light chain variable region (VL) of SEQ ID NO:2.

In some embodiments, the patient has MAS or HLH. In some embodiments, the patient is at risk of developing MAS or HLH. In some embodiments, the HLH/MAS patient or patient at risk of developing HLH/MAS has a disease or condition such as systemic-onset juvenile idiopathic arthritis (SoJIA), systemic lupus erythematosus (SLE), Kawasaki disease, or adult-onset Still's disease.

“Systemic-onset juvenile idiopathic arthritis (SoJIA)” (or the juvenile onset form of Still's disease) is a type of juvenile idiopathic arthritis (JIA) with extra-articular manifestations like fever and rash apart from arthritis. It was originally called systemic-onset juvenile rheumatoid arthritis or Still's disease. Predominantly extra-articular manifestations like high fevers, rheumatic rash, enlargement of the liver and spleen, enlargement of the lymph nodes, and anemia. Other manifestations include inflammation of the pleura, inflammation of the pericardium, inflammation of the heart's muscular tissue, and inflammation of the peritoneum are also seen.

“Systemic lupus erythematosus (SLE)” is an autoimmune disease in which the body's immune system mistakenly attacks healthy tissue in many parts of the body. Symptoms vary among people and may be mild to severe. Common symptoms include painful and swollen joints, fever, chest pain, hair loss, mouth ulcers, swollen lymph nodes, feeling tired, and a red rash which is most commonly on the face. Often there are periods of illness, called flares, and periods of remission during which there are few symptoms.

“Kawasaki disease” is a syndrome of unknown cause that results in a fever and mainly affects children under 5 years of age. It is a form of vasculitis, where blood vessels become inflamed throughout the body. The fever typically lasts for more than five days and is not affected by usual medications. Other common symptoms include large lymph nodes in the neck, a rash in the genital area, lips, palms, or soles of the feet, and red eyes. Within three weeks of the onset, the skin from the hands and feet may peel, after which recovery typically occurs. In some children, coronary artery aneurysms form in the heart.

“Adult-onset Still's disease (AOSD)” a rare systemic autoinflammatory disease characterized by the classic triad of fevers, joint pain, and a distinctive salmon-colored bumpy rash. The disease is considered a diagnosis of exclusion. Levels of the iron-binding protein ferritin may be extremely elevated with this disorder.

In some embodiments, the patient has an abnormally high blood ferritin level. In some embodiments, the blood ferritin level is at least 300 ng/ml, or at least 350 ng/ml, 400 ng/ml, 450 ng/ml, 500 ng/ml, 550 ng/ml, 600 ng/ml, 650 ng/ml, 684 ng/ml, 700 ng/ml, 720 ng/ml, 740 ng/ml, 750 ng/ml, 760 ng/ml, 784 ng/ml, 800 ng/ml, 820 ng/ml, 840 ng/ml, 850 ng/ml, 860 ng/ml, 884 ng/ml, 900 ng/ml, 950 ng/ml, or 1000 ng/ml.

In some embodiments, administration of anti-GM-CSF antibody provided herein decreases the blood ferritin level by at least 10%, at least 20%, at least 30%, at least 50% or at least 70%, as compared to untreated control. In some embodiments, administration of anti-GM-CSF antibody provided herein reverses the abnormally high blood ferritin level in the patient.

In some embodiments, the patient has an abnormally low hemoglobin level. In some embodiments, the hemoglobin level is lower than 11 g/dL, or lower than 10.5 g/dL, 10 g/dL, 9.5 g/dL, 9 g/dL, 8.5 g/dL, 8 g/dL, 7.5 g/dL, or 7 g/dL.

In some embodiments, administration of anti-GM-CSF antibody provided herein increases the blood hemoglobin level by at least 0.2, 0.3, 0.4, 0.5, 1, 1.5, 2, 3, 4, 5, 6, 7, 8-fold, as compared to untreated control. In some embodiments, administration of anti-GM-CSF antibody provided herein reverses the abnormally low hemoglobin level in the patient.

In some embodiments, the patient has an abnormally low platelet count. In some embodiments, the platelet count is lower than 140×10⁹/L, or lower than 130×10⁹/L, 120×10⁹/L, 110×10⁹/L, 100×10⁹/L, 90×10⁹/L, 80×10⁹/L, 70×10⁹/L, or 60×10⁹/L.

In some embodiments, administration of anti-GM-CSF antibody provided herein increases the platelet count by at least 0.2, 0.3, 0.4, 0.5, 1, 1.5, 2, 3, 4, 5, 6, 7, 8-fold, as compared to untreated control. In some embodiments, administration of anti-GM-CSF antibody provided herein reverses the abnormally low platelet count in the patient.

In some embodiments, the patient has an abnormally low neutrophil count. In some embodiments, the neutrophil count is lower than 1.4×10⁹/L, or lower than 1.3×10⁹/L, 1.2×10⁹/L, 1.1×10⁹/L, 1.0×10⁹/L, 0.9×10⁹/L, 0.8×10⁹/L, 0.7×10⁹/L, or 0.6×10⁹/L.

In some embodiments, administration of anti-GM-CSF antibody provided herein increases the neutrophil count by at least 0.1, 0.2, 0.3, 0.4, 0.5, 1, 1.5, 2, 3, 4, 5, 6, 7, 8-fold, as compared to untreated control. In some embodiments, administration of anti-GM-CSF antibody provided herein reverses the abnormally low neutrophil count in the patient.

In some embodiments, the patient has an abnormally high fasting triglyceride level. In some embodiments, the fasting triglyceride level is higher than 2.49 mmol/L, or higher than 2.55 mmol/L, 2.6 mmol/L, 2.66 mmol/L, 2.72 mmol/L, 2.77 mmol/L, 2.83 mmol/L, 2.89 mmol/L, 2.94 mmol/L, 3 mmol/L, 3.06 mmol/L, 3.11 mmol/L, 3.17 mmol/L, 3.23 mmol/L, 3.28 mmol/L, 3.34 mmol/L, or 3.4 mmol/L. In some embodiments, the fasting triglyceride level is higher than 220 mg/dl, or higher than 225 mg/dl, 230 mg/dl, 235 mg/dl, 240 mg/dl, 245 mg/dl, 250 mg/dl, 255 mg/dl, 260 mg/dl, 265 mg/dl, 270 mg/dl, 275 mg/dl, 280 mg/dl, 285 mg/dl, 290 mg/dl, 295 mg/dl, or 300 mg/dl.

In some embodiments, administration of anti-GM-CSF antibody provided herein decreases the fasting triglyceride level by at least 10%, at least 20%, at least 30%, at least 50% or at least 70%, as compared to untreated control. In some embodiments, administration of anti-GM-CSF antibody provided herein reverses the abnormally high fasting triglyceride level in the patient.

In some embodiments, the patient has an abnormally low blood fibrinogen level. In some embodiments, the blood fibrinogen level is lower than 1.5 g/L, or lower than 1.4 g/L, 1.3 g/L, 1.2 g/L, 1.1 g/L, 1.0 g/L, 0.9 g/L, 0.8 g/L, 0.7 g/L, 0.6 g/L, or 0.5 g/L.

In some embodiments, administration of anti-GM-CSF antibody provided herein increased the blood fibrinogen level by at least 0.1, 0.2, 0.3, 0.4, 0.5, 1, 1.2, 1.5, 2, 3, 4, 5, 6, 7, 8-fold in the patient. In some embodiments, administration of anti-GM-CSF antibody provided herein reverses the abnormally low blood fibrinogen level in the patient.

In some embodiments, administration of anti-GM-CSF antibody provided herein decreases or reverses the high hemophagocytosis level in the patient.

In some embodiments, administration of anti-GM-CSF antibody provided herein decreases or reverses the high inflammatory infiltration level in the patient.

In some embodiments, administration of anti-GM-CSF antibody provided herein decreases or reverses the high lymphocyte exhaustion in the patient.

In some embodiments, administration of anti-GM-CSF antibody provided herein increases the bone marrow viable cells.

In some embodiments, the patient has macrophage activation. In some embodiments, the patient has an elevated soluble CD163 level as compared to a healthy individual. In some embodiments, the patient has lymphocyte activation. In some embodiments, the patient has an elevated soluble IL-2 receptor level as compared to a healthy individual.

In some embodiments, the patient meets two or more of the above standards. In some embodiments, the patient meets three, four, five or more of the above standards.

The amount of the antibodies of the disclosure which will be effective in the treatment. The precise dose to be employed in the formulation will also depend on the route of administration, and the seriousness of the disease, disorder or condition, and should be decided according to the judgment of the practitioner and each patient's circumstances.

Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In some embodiments, the dose for IMH001 is from 0.1 to 25 mg/kg, from 0.3 to 20 mg/kg, from 0.3 to 15 mg/kg, from 0.3 to 10 mg/kg, from 0.5 to 20 mg/kg, from 1 to 20 mg/kg, from 5 to 20 mg/kg, from 6 to 20 mg/kg, from 6 to 10 mg/kg, from 5 to 10 mg/kg per administration. In some embodiments, the dose is at least 0.3 mg/kg, or at least 0.6 mg/kg, 1 mg/kg, 1.3 mg/kg, 1.6 mg/kg, 2 mg/kg, 2.3 mg/kg, 2.6 mg/kg, 3 mg/kg, 4 mg/kg, 5 mg/kg, 6 mg/kg, 7 mg/kg, 8 mg/kg, 9 mg/kg, or 10 mg/kg. In some embodiments, the dose is not higher than 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, or 2 mg/kg. In some embodiments, the administration dose is 2 mg/kg, 3 mg/kg, 4 mg/kg, 5 mg/kg, 6 mg/kg, 7 mg/kg, 8 mg/kg, 9 mg/kg, 10 mg/kg, 12 mg/kg, 15 mg/kg, 20 mg/kg, or 25 mg/kg.

In some embodiments, the administration is once a day, once every 2 days, once every 3 days, once every 4 days, once a week, twice a week, once every two weeks, once every three weeks, once a month, or once every two months.

Methods of administration of the antibodies include but are not limited to intradermal, intramuscular, intraperitoneal, intravenous, subcutaneous, intranasal, epidural, and oral routes. The antibody or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antibody of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antibody or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

In another embodiment, the antibody or composition can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the antibody or composition can be delivered in a controlled release system. In one embodiment, a pump may be used (see Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

### Combinations and Formulations

In some embodiments, the patient can be further administered a secondary agent. Non-limiting examples of secondary agent include glucocorticoids, cyclosporine and anakinra.

Examples of glucocorticoids include cortisol (hydrocortisone), cortisone, prednisone, prednisolone, methylprednisolone, dexamethasone, betamethasone, triamcinolone, deflazacort, fludrocortisone acetate, deoxycorticosterone acetate, aldosterone, and beclometasone.

The antibody which is formulated is preferably essentially pure and desirably essentially homogeneous (e.g., free from contaminating proteins etc.). “Essentially pure” antibody means a composition comprising at least about 90% by weight of the antibody, based on total weight of proteins in the composition, preferably at least about 95% by weight, “Essentially homogeneous” antibody means a composition comprising at least about 99% by weight of antibody, based on total weight of proteins in the composition.

The term “pharmaceutical formulation” refers to a preparation that contains an anti-GM-CSF antibody in such form as to permit the biological activity of the antibody to be effective, and which contains no additional components which are unacceptably toxic to a subject to which the formulation would be administered.

The formulation can be a liquid or aqueous. Liquid formulations are aqueous solutions or suspensions, prepared in a suitable aqueous solvent, such as water or an aqueous/organic mixture, such as water alcohol mixtures.

In certain embodiments, the liquid pharmaceutical formulation comprises 20 mg/ml-200 mg/ml antibody, e.g., 30 mg/ml-200 mg/ml, 40 mg/ml-200 mg/ml, 50 mg/ml-200 mg/ml, 60 mg/ml-200 mg/ml, 70 mg/ml-200 mg/ml, 80 mg/ml-200 mg/ml, 90 mg/ml-200 mg/ml, 100 mg/ml-200 mg/ml, 110 mg/ml-200 mg/ml, 120 mg/ml-200 mg/ml, 130 mg/ml-200 mg/ml, 140 mg/ml-200 mg/ml, 150 mg/ml-200 mg/ml, 160 mg/ml-200 mg/ml, 170 mg/ml-200 mg/ml, 180 mg/ml-200 mg/ml, 190 mg/ml-200 mg/ml, 20 mg/ml-190 mg/ml, 20 mg/ml-180 mg/ml, 20 mg/ml-170 mg/ml, 20 mg/ml-160 mg/ml, 20 mg/ml-150 mg/ml, 20 mg/ml-140 mg/ml, 20 mg/ml-130 mg/ml, 20 mg/ml-120 mg/ml, 20 mg/ml-110 mg/ml, 20 mg/ml-100 mg/ml, 20 mg/ml-90 mg/ml, 20 mg/ml-80 mg/ml, 20 mg/ml-70 mg/ml, 20 mg/ml-60 mg/ml, 20 mg/ml-50 mg/ml, 20 mg/ml-40 mg/ml, 30 mg/ml-190 mg/ml, 40 mg/ml-180 mg/ml, 50 mg/ml-170 mg/ml, 60 mg/ml-160 mg/ml, 70 mg/ml-150 mg/ml, 80 mg/ml-140 mg/ml, 90 mg/ml-130 mg/ml, 100 mg/ml-120 mg/ml. In certain embodiments, the liquid pharmaceutical formulation comprises 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, or 200 mg/ml antibody.

In another embodiment the pharmaceutical formulation further comprises additional excipients. The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity.

“Excipients” includes, but is not limited to, stabilizers, for example, human serum albumin (HSA), bovine serum albumin (BSA), α-casein, globulins, α-lactalbumin, LDH, lysozyme, myoglobin, ovalbumin, RNase A; buffering agents, for example, citric acid, HEPES, PBS, histidine, potassium acetate, potassium citrate, potassium phosphate (KH₂PO₄), sodium acetate, sodium bicarbonate, sodium citrate, sodium phosphate (NaH₂PO₄), Tris base, and Tris-HCl; amino acids/metabolites, for example, glycine, alanine (α-alanine, β-alanine), arginine, betaine, leucine, lysine, glutamic acid, aspartic acid, histidine, proline, 4-hydroxyproline, sarcosine, γ-aminobutyric acid (GABA), opines (alanopine, octopine, strombine), and trimethylamine N-oxide (TMAO); surfactants, for example, polysorbate 20 and 80, and poloxamer 407; lipid molecules, for example, phosphatidyl choline, ethanolamine, and acethyltryptophanate: polymers, for example, polyethylene glycol (PEG), and polyvinylpyrrolidone (PVP) 10, 24, 40; low molecular weight excipients, for example, arabinose, cellobiose, ethylene glycol, fructose, fucose, galactose, glycerin/glycerol, glucose, inositol, lactose, maltose, maltotriose, mannose, melibiose, 2-methyl-2,4-pentanediol, octulose, propylene glycol, raffinose, ribose, sucrose, trehalose, xylitol, and xylose; and high molecular weight excipients, for example, cellulose, β-cyclodextrin, dextran (10 kd), dextran (40 kd), dextran (70 kd), ficoll, gelatin, hydroxypropylmethyl-cellulose, hydroxyethyl starch, maltodextrin, methocel, PEG (6 kd), poly dextrose, polyvinylpyrrolidone (PVP) k15 (10 kd), PVP (40 kd), PVP k30 (40 kd), PVP k90 (1000 kd), sephadex G-200, and starch; antioxidants, for example, ascorbic acid, cysteine HCl, thioglycerol, thioglycolic acid, thiosorbitol, and glutathione; reducing agents, for example, cysteine HCl, dithiothreotol, and other thiol or thiophenes; chelating agents, for example, EDTA, EGTA, glutamic acid, and aspartic acid; inorganic salts/metals, for example, Ca²⁺, Ni²⁺, Mg²⁺, Mn²⁺, Na₂SO₄, (NH₄)₂SO₄, Na₂HPO₄/NaH₂PO₄, K₂HPO₄/KH₂PO₄, MgSO₄, and NaF; organic salts, for example, Na acetate, Na polyethylene, Na caprylate (Na octanoate), proprionate, lactate, succinate, and citrate; organic solvents, for example, acetonitrile, dimethylsulfoxide (DMSO), and ethanol. For additional information regarding excipients, see Remington's Pharmaceutical Sciences (by Joseph P. Remington, 18th ed., Mack Publishing Co., Easton. Pa.), which is incorporated herein in its entirety.

By “isotonic” is meant that the formulation of interest has essentially the same osmotic pressure as human blood. Isotonic formulations generally have an osmotic pressure from about 250 to about 350 mOsm (e.g., from about 250 to about 340, from about 250 to about 330, from about 250 to about 320, from about 250 to about 310, from about 250 to about 300, from about 260 to about 350, from about 270 to about 350, from about 280 to about 350, from about 290 to about 350, from about 300 to about 350, from about 260 to about 340, from about 270 to about 330, from about 280 to about 320, from about 290 to about 310 mOsm). Isotonicity can be measured using a vapor pressure or ice-freezing type osmometer, for example. In certain embodiments, the isotonicity is about 300 mOsm. A isotonicity modifier can be one or more selected from the group consisting of sucrose, trehalose, mannitol, arginine, and sodium chloride. In certain embodiments, the isotonicity modifier is of a range of about 130-250 mM (e.g., from about 130 mM to about 240 mM, from about 150 mM to about 240 mM, from about 180 mM to about 240 mM, from about 200 mM to about 240 mM, from about 130 mM to about 220 mM, from about 150 mM to about 220 mM, from about 180 mM to about 220 mM, from about 130 mM to about 210 mM, from about 150 mM to about 210 mM, from about 180 mM to about 210 mM, from about 200 mM to about 220 mM, from about or 200 to about 210 mM). In certain embodiments, the isotonicity modifier is about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM. In certain embodiments, the isotonicity modifier comprises sucrose or trehalose.

As used herein, “pH buffer” refers to a buffered solution that resists changes in pH by the action of its acid-base conjugate components. The buffer of this disclosure preferably has a pH in the range from about 4.5 to about 7.5, preferably from about 5.0 to about 7.0, for example from about 5.0 to about 6.9, about 5.2 to about 6.8, about 5.3 to about 6.7, about 5.4 to about 6.6, about 5.5 to about 6.5, about 5.6 to about 6.4, about 5.7 to about 6.3, about 5.8 to about 6.2, about 5.9 to about 6.1, about 5.5 to about 6.4, about 5.5 to about 6.3, about 5.5 to about 6.2, about 5.5 to about 6.1, or about 5.5 to about 6.0. In one embodiment the buffer has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0. In one embodiment the buffer has a pH 5.8. Various means may be utilized in achieving the desired pH level, including, but not limited to the addition of the appropriate buffer.

In certain embodiments, the pH buffer comprises histidine, acetate, citrate, and succinate. In certain embodiments, the pH buffer comprises from about 10 to about 30 mM histidine and/or from about 10 to about 30 mM acetate, for example, from about 10 to about 25 mM, from about 10 to about 20 mM, or from about 15 mM to about 20 mM histidine and/or from about 10 to about 25 mM, from about 10 mM to about 20 mM, or from about 15 mM to about 20 mM acetate. In certain embodiments, the pH buffer comprises about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM histidine or acetate.

In certain embodiments, the pH buffer comprises L-histidine and histidine hydrochloride monohydrate. In certain embodiments, the pH buffer comprises 20 mM histidine comprising about 4.4 mM L-histidine and about 15.6 mM histidine hydrochloride monohydrate, about 5.2 mM L-histidine and about 14.8 mM histidine hydrochloride monohydrate, about 6.2 mM L-histidine and about 13.8 mM histidine hydrochloride monohydrate, about 6.8 mM L-histidine and about 13.2 mM histidine hydrochloride monohydrate, about 7.8 mM L-histidine and about 12.2 mM histidine hydrochloride monohydrate, about 8.4 mM L-histidine and about 11.6 mM histidine hydrochloride monohydrate, or about 9 mM L-histidine and about 11 mM histidine hydrochloride monohydrate.

As used herein, a “surfactant” refers to a surface-active agent, preferably a nonionic surfactant. Examples of surfactants herein include polysorbate (for example, polysorbate 20, polysorbate 40, polysorbate 60, polysorbate 65, polysorbate 80, and, polysorbate 85); poloxamer (e.g., poloxamer 188 and poloxamer 407); Triton; sodium dodecyl sulfate (SDS); sodium laurel sulfate; sodium octyi glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamido propyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g., Piuronics, PF68 etc); etc.

The surfactant concentration is generally from about 0.0001% (w/v) to about 1.0% (w/v), from about 0.010% (w/v) to about 0.5% (w/v), for example, from about 0.015% (w/v) to about 0.03% (w/v), from about 0.02% (w/v) to about 0.03% (w/v), from about 0.025% (w/v) to about 0.03% (w/v), from about 0.01% (w/v) to about 0.025% (w/v), from about 0.01% (w/v) to about 0.02% (w/v), or from about 0.01% (w/v) to about 0.015% (w/v). In one embodiment, the surfactant provided herein comprises polysorbate 80 or polysorbate 20. In certain embodiments, the surfactant comprises about 0.01% (w/v), 0.015% (w/v), 0.02% (w/v), 0.025% (w/v), or 0.03% (w/v) polysorbate 80 or polysorbate 20. In certain embodiments, the surfactant comprises about 0.02% (w/v) polysorbate 80.

In certain embodiments, the liquid pharmaceutical formulation further comprises antioxidant, preservatives, or mixtures thereof.

The term “antioxidant” refers to an agent that inhibits the oxidation of other molecules. Examples of the antioxidant include ascorbic acid, citrate, lipoic acid, uric acid, cysteine HCl, monothioglycerol, thioglycerol, thioglycolic acid, thiosorbitol, tocopherol, carotene, lycopene and glutathione; reducing agents, for example, cysteine HCl, dithiothreotol, phosphonate compounds, e.g., etidronic acid, desferoxamine and malate, and other thiol or thiophenes and methionine. In other embodiments the antioxidant is a metal chelator. Metal chelators include, but are not limited to ethylenediaminetetraacetate (“EDTA”), ethylene glycol tetra acetic acid (“EGTA”), (thiamine tetrahydrofurfuryl disulfide (“TTFD”), and 2,3-dimercaptosuccinic acid (“DMSA”). In certain embodiments the formulation comprises about 1 mM to about 50 mM antioxidant. In one embodiment the formulation comprises about 5 mM, about 10 mM, about 15 mM, about 20 mM, about 25 mM, about 30 mM, about 35 mM, about 40 mM, or about 45 mM antioxidant.

The term “preservative” refers to pharmaceutically acceptable excipients which prevent the growth of micro-organisms within the composition. More particularly, the disclosure provides a preservative containing multi-dose liquid composition which protects the composition against microbial contamination.

In one embodiment, the preservative is present within the composition in an amount of between 0.001% (w/v) to 2% (w/v). In one embodiment, the preservative is present within the composition in an amount of between 0.002% (w/v) to 1% (w/v). In one embodiment, the one or more preservative is selected from phenol, m-cresol, benzyl alcohol, chlorobutanol, ethanol, phenoxyethanol, p-chlor-m-cresol, methyl paraben, propyl paraben, benzalkonium chloride, thiomersal or any combinations thereof. In one embodiment, the one or more preservative is selected from phenol, m-cresol, benzyl alcohol and chlorobutanol.

The viscosity of an anti-GM-CSF antibody formulation can be controlled for subcutaneous, intravenous or intramuscular administration. The viscosity can be affected by protein concentration and pH. For example, as the protein concentration increases, the viscosity can increase. An increase in pH can decrease the viscosity of the anti-GM-CSF antibody formulation. In some protein formulations, sodium chloride is added to reduce the viscosity of the formulation. Additional components that can affect viscosity of an anti-GM-CSF antibody formulation are amino acids such as histidine and arginine.

The liquid pharmaceutical formulations described herein can have various viscosities. Methods of measuring viscosity of liquid pharmaceutical formulations are known to those in the art, and can include, e.g., a rheometer (e.g., Anton Paar MCR301 Rheometer with either a 50 mm, 40 mm or 20 mm cone accessory). In some embodiments of the present disclosure, the viscosities were reported at a high shear limit of 1000 per second shear rate. In some embodiments, the liquid pharmaceutical formulations has a viscosity between 1.0 cP=L10% and 20 cP=10%. In some embodiments, the liquid pharmaceutical formulations has a viscosity of less than 20 cP, less than 18 cP, less than 15 cP, less than 13 cP, or less than 11 cP. One of skill in the art will appreciate that viscosity is dependent on temperature, thus, unless otherwise specified, the viscosities provided herein are measured at 25° C. unless otherwise specified. In some embodiments, the viscosity of the liquid pharmaceutical formulations is 1.0 cP±10%, 2.0 cP±10%, 3.0 cP±L10%, 3.1 cP±10%, 3.2 cP±10%, 3.5 cP±10%, 3.6 cP±10%, 3.8 cP±10%, 4.0 cP±10%, 5.0 cP±10%, 5.3 cP±10%, 6.0 cP±10%, 6.3 cP±10%, 6.4 cP±10%, 6.8 cP±10%, 7.0 cP±10%, 7.1 cP±10%, 7.4 cP±10%, 8.0 cP±10%, 9.0 cP±L10%, 10.0 cP±10%, 11.0 cP±10%, 12.0 cP±10%, 13.0 cP±10%, 14.0 cP±10%, 15.0 cP±10%, or 16 cP±10% at 25° C..

In certain embodiments, the liquid pharmaceutical formulation further comprises a viscosity modifier. In one embodiment, the viscosity modifier is an amino acid. In one embodiment, the viscosity modifier is L-proline. In certain embodiments, the viscosity modifier is at a concentration of from 1%0%±0.2% to 5%1% w/v. In one embodiment, the viscosity modifier is proline at a concentration of 1.5%±0.3% or about 1.5%. In one embodiment, the viscosity modifier is proline at a concentration of 3%±0.6%, or about 3%.

In a first aspect, the present disclosure provides a novel liquid pharmaceutical formulation, comprising:
a) an anti-GM-CSF antibody of a concentration of 20 mg/ml-200 mg/ml as the antibody;
b) acetate or histidine in a concentration of 10 mM-30 mM as the buffering agent; c) sucrose or trehalose in a concentration of 130 mM-250 mM as the isotonicity modifier;
d) Polysorbate 80 or Polysorbate 20 in a concentration of 0.01% (w/v)-0.03% (w/v), as surfactant;
   wherein the formulation has a pH of about 4.5 to about 7.5, preferably, about 5.5 to about 6.1.

In a further embodiment, wherein the formulation does not comprise further excipient.

In a preferred embodiment, the present disclosure provides a novel liquid pharmaceutical formulation, comprising:
a) anti-GM-CSF antibody of a concentration of 50 mg/ml-150 mg/ml;
b) histidine in a concentration of 10 mM-20 mM;
c) sucrose in a concentration of 200 mM-220 mM;
d) Polysorbate 80 in a concentration of 0.01% (w/v)-0.03% (w/v);
   wherein the formulation has a pH of 5.5 to 6.1.

In a further embodiment, wherein the formulation does not comprise further excipient.

In a more preferred embodiment, the present disclosure provides a novel liquid pharmaceutical formulation, comprising:
a) anti-GM-CSF antibody of a concentration of 100 mg/ml, wherein the anti-GM-CSF antibody comprises a heavy chain variable region CDR1 (HCDR1) of SEQ ID NO: 1, a HCDR2 of SEQ ID NO: 2, a HCDR3 of SEQ ID NO: 3, a light chain variable region CDR1(LCDR1) of SEQ ID NO: 4, a LCDR2 of SEQ ID NO: 5, and a LCDR3 of SEQ ID NO: 6;
b) histidine in a concentration of 20 mM;
c) sucrose in a concentration of 220 mM;
d) Polysorbate 80 in a concentration of 0.02% (w/v);
   wherein the formulation has a pH of 5.8.

In a further embodiment, wherein the formulation does not comprise further excipient.

The pharmaceutical formulations can be administered to a patient by parenteral routes such as injection (e.g., subcutaneous, intravenous, intramuscular, intraperitoneal, etc.) or percutaneous, mucosal, nasal, pulmonary or oral administration. Numerous reusable pen or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (sanofi-aventis, Frankfurt, Germany). Examples of disposable pen or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif), the PENLET™ (Haselmeier, Stuttgart, Germany), the EPIPEN (Dey, L. P.), and the HUMIRA™ Pen (Abbott Labs, Abbott Park, Ill.).

The use of a microinfusor to deliver the pharmaceutical formulations of the present disclosure is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) or viscous solutions.

In certain embodiments, the present disclosure provides a prefilled syringe comprising any of the liquid formulations described herein. In certain embodiments, the syringe is a 1 mL or 2.25 mL long glass syringe filled with a 27-gauge thin wall needle, a fluorocarbon coated rubber plunger and a rubber needle shield.

In one aspect, the liquid pharmaceutical formulations of the present disclosure can be kept at room temperature, refrigerated (e.g., 2-8° C.), or frozen (e.g., −20° C. or −70° C.) for storage.

In certain embodiments, the formulation of any of the preceding aspects has an attribute selected from the group consisting of: (i) the formulation is stable to long-term storage at 50° C., 40° C., 25° C., 5° C., −20° C., −30° C., and −80° C.; (ii) the formulation is low-viscosity (viscosity less than 10 cP); (iv) the formulation is iso-osmolar to physiologic conditions; (v) the formulation is stable to and compatible with intravenous or subcutaneous delivery devices and procedures; and (vi) the formulation is stable to long-term storage in a glass vial or in a prefilled syringe.

In one aspect, the formulation substantially retains its physical and chemical stability, as well as its biological activity upon storage. The storage period is generally selected based on the intended shelf-life of the formulation. Various analytical techniques for measuring protein stability are available in the art and are reviewed, for example, in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. For example, the liquid formulation is stable at about 40° C. for at least about 3 days, 5 days, 1 week, 2 weeks, 3 weeks, 4 weeks, 5 weeks or 6 weeks. The liquid formulation in another aspect is stable at about 5° C. and/or 25° C. for at least about 1 month, at least about 3 months, at least about 6 months, at least about 9 months, at least about 12 months, at least about 18 months, at least about 24 months, at least about 30 months, or at least about 36 months; and/or stable at about −20° C. and/or −70° C. for at least about 1 month, at least about 3 months, at least about 6 months, at least about 9 months, at least about 12 months, at least about 18 months, at least about 24 months, at least about 30 months, at least about 36 months, at least about 42 months, or at least about 48 months. Furthermore, the liquid formulation may, in some embodiments, be stable following freezing (to, e.g., −80° C.) and thawing, for example following 1, 2 or 3 cycles of freezing and thawing.

The stability of a liquid formulation can be evaluated qualitatively and/or quantitatively in a variety of different ways, including evaluation of dimer, multimer and/or aggregate formation (for example using size exclusion chromatography (SEC), matrix-assisted laser desorption-ionization time-of-flight mass spectrometry (MALDI-TOF MS), analytical ultracentrifugation, light scattering (photon correlation spectroscopy, dynamic light scattering (DLS), static light scattering, multi-angle laser light scattering (MALLS)), flow-based microscopic imaging, electronic impedance (coulter) counting, light obscuration or other liquid particle counting system, by measuring turbidity, and/or by visual inspection); by assessing charge heterogeneity using cation exchange chromatography (CEX), isoelectric focusing (IEF), e.g., capillary technique (cIEF), or capillary zone electrophoresis; amino-terminal or carboxy-terminal sequence analysis; mass spectrometric analysis; SDS-PAGE or SEC analysis to compare fragmented, intact and multimeric (i.e., dimeric, trimeric, etc.) antibody; peptide map (for example tryptic or LYS-C) analysis; evaluating biological activity or antigen binding function of the antibody; and the like. Stability of a solid-state formulation can also be evaluated qualitatively and/or quantitatively in a variety of different ways, including direct tests, such as identifying crystal structure by X-Ray Powder Diffraction (XRPD); evaluating antibody structure in the solid state using Fourier Transform Infrared Spectroscopy (FTIR); and measuring thermal transitions in the lyophilized solid (melting, glass transition, etc.) using Differential Scanning calorimetry (DSC) and indirect tests such as measuring moisture content by Karl Fisher test, e.g., to extrapolate the likelihood of chemical instability through hydrolysis. Instability may involve any one or more of: aggregation (e.g., non-covalent soluble aggregation, covalent soluble aggregation (e.g., disulfide bond rearrangement/scrambling), insoluble aggregation), deamidation (e.g., Asn deamidation), oxidation (e.g., Met oxidation), isomerization (e.g., Asp isomeriation), clipping/hydrolysis/fragmentation (e.g., hinge region fragmentation), succinimide formation, N-terminal extension, C-terminal processing, glycosylation differences, and the like.

### EXAMPLES

### Example 1. IMH001 Inhibited MAS Development and Ameliorated Symptoms

This study is performed to evaluate the in vivo efficacy of IMH001 (see sequences in Table 1), a GM-CSF neutralization antibody, on delaying the disease progression in a macrophage activation syndrome (MAS) model (induced by CBMC transplantation) in NOG-EXL mice. This lymphocyte-independent, effector-driven MAS mice model can be established by engrafting human umbilical cord blood (UCB) into NOG-EXL mice. NOG-EXL (NOD.Cg-Prkdc^{scid}IL2g^{tm1Sug}Tg (SV40/HTLV-IL3, CSF2) is a gene modified strain based on NOG (NOD.Cg-Prkdc^{scid}IL2^{tm1Sug}) mice, which is immunodeficient and overexpress human IL-3 and GM-CSF continuously, which can enrich the myeloid cell populations, including Dc, monocyte and macrophage, following human stem cell engraftment. A total of 46 NOG-EXL and 8 NOG mice were used in the study.

TABLE 1
VH/VL sequences of IMH001

| | Sequence | SEQ ID NO: |
|---|---|---|
| VH | EVQLVQSGAEVKKPGASVKVSCKASGYTFTSHYLHWVRQAPGQGLEWMGWIFPGDD | 1 |
| | KTKYNEKFKGRVTMTSDTSISTAYMELSRLRSDDTAVYYCARGTKYLNWNFDVWGQ | |
| | GTTVTVSS | |
| VL | DIQLTQSPSFLSASVGDRVTITCKANQNVGTTLAWYQQKPGKSPKALIYSASYRYS | 2 |
| | GVPDRFSGSGSGTDFTLTISSLQPEDFATYFCHQYTTYPLTFGGGTKVEIK | |

The model showed progressive disease with many of the hallmarks of MAS and rapidly developed a progressive anemia without the need for exogenous immune stimulation. Therefore, the model can be used to examine the contributions of the monocyte/macrophage arm and identify new therapeutic strategies to MAS/HLH.

### 1.1 Method

Five to six-week-old NOG-EXL mice were irradiated with 1 Gy before tail vein injection of OKT3-pretreated cord blood mononuclear cells (CBMC) reconstitution. The day of reconstruction was defined as Day 0. OKT3-pretreated CBMC engrafted NOG-EXL mice were treated with biweekly i.p. doses of PBS or 10 mg/kg IMH001 starting from week 1 of engraftment. Animal body weights, CBC counting, and serum ferritin were measured at indicated time points.

Four NOG mice (G1) and 14 NOG-EXL mice (6 mice for G2 and 8 mice for G3, respectively) were dosed one week after reconstruction (Day 7), twice a week for 15 weeks. The grouping and dosing regimens were as follows in Table 2:

TABLE 2
Grouping and Dosing Regimen

| | | | | Dose | Volume | | |
|---|---|---|---|---|---|---|---|
| Strain | Group | Test Article | N | (mg/kg) | (μL/g) | Regimen | Remarks |
| NOG | 1 | Vehicle | 4 | N/A | 10 | i.p., | Dosing |
| | | (saline) | | | | BIW*15W | initiation: Day 7 (1 |
| NOG- | 2 | Vehicle | 6 | N/A | | i.p., | week after |
| EXL | | (saline) | | | | BIW*15W | reconstruction); |
| | 3 | IMH001 | 8 | 10 | | i.p., | Experimental |
| | | | | | | BIW*15W | endpoint: Day 112 |
| | | | | | | | (16 weeks after |
| | | | | | | | reconstruction) |

Body weight: The body weight of mice was measured and recorded once a week after CBMC transplantation.

CBC counting: Whole blood was collected from mice and counted by a CBC machine at week 4, 8 and 16. And at the end of the study, i.e. week 16, total bone marrow viable cells were also counted. Before blood collection, mice were anesthetized with 3-4% isoflurane.

Cytokines by MSD: Serum was collected for IL-10, IL-6, TNF-α, GM-CSF, IFN-γ and IL-12 at week 8, and 10. Before blood collection, mice were anesthetized with 3-4% isoflurane.

Ferritin, fibrinogen and triglycerides: Serum was collected from mice at week 8 and 16. Then measured separately using ELISA kits. Prior to blood collection, mice were anesthetized with 3-4% isoflurane.

Experimental endpoint collection: The endpoint is Day 112/Week 16. Spleens were collected and weighed from all mice of each groups; cell smears from bone marrow, sections of liver and spleen were collected for Wright Giemsa staining to detect the presence of hemophagocytosis. Activated lymphoid tissue, spleen/lymph nodes, liver, and meninges were collected for H&E section to detect inflammatory cell infiltration and depletion of lymphoid follicles in spleen/lymph nodes.

### Statistics

All data statistical analysis methods involved in this project needed to be consistent with the protocol, and the results were expressed as mean±SEM. Firstly, F-test (IDBS E-WorkBook 10.5.0 (64 bit) © TDBS) was used to analyze the homogeneity of variance. If the variance was homogeneity (p>0.05), one-way ANOVA was carried out. T-test was used for two groups comparation, and Dunnett's multiple comparison method was used for multiple groups comparation. If the variance was heterocedasticity (p≤0.05), Kruskal Wallis method was performed for nonparametric test, U-test was used for two groups comparation (Kruskal Wallis parameter test has significant difference, p≤0.05), and Dunn's multiple comparison method was used for multiple groups comparation (Kruskal Wallis parameter test has no significant difference, p>0.05). p<0.05 was considered as significant difference.

### 1.2 Results

Shortly after OKT3-pretreated CBMC engraftment into NOG-EXL mice, the mice developed MAS characterized with body weight loss, a progressive drop in red blood cell (RBC) count, hemoglobin (HGB) and hematocrit (HCT), and hyperferritin.

### 1.2.1 Body Weight Change

The treatment with IMH001, however, completely reversed the disease development.

Animals were weighed weekly after CBMC engraftment.

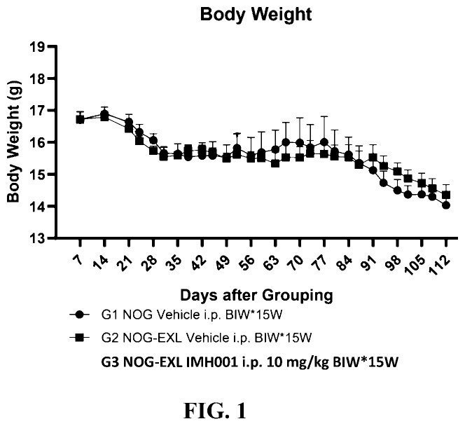

No obvious abnormalities were found in each group during the experiment. The body weight changes at each time point in each group are shown in FIG. 1. As the disease progressed, there was continuous body weight loss observed in CBMC transplanted NOG or NOG-EXL mice. Compared with the vehicle control group (G2), the body weight of IMH001 treatment groups (G3: 10 mg/kg) was significantly increased from Day 63 to Day 112 (p<0.05). This result indicates that IMH001 treatment effectively improved animal body weight loss caused by MAS.

### 1.2.2 Peripheral Blood and Bone Marrow Cell Counting

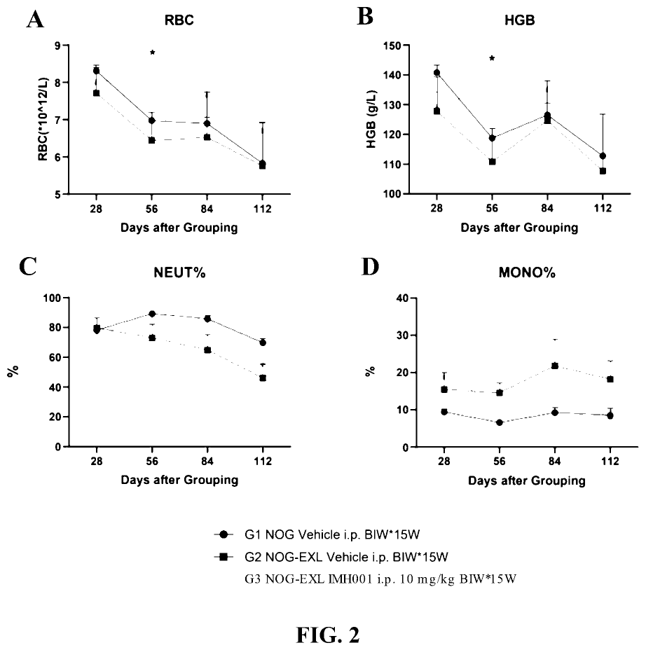

Whole blood was collected from mice and complete blood count (CBC) analysis was performed at week 4, 8, 12 and 16. At the study end (week 16), total bone marrow viable cells were also counted. The RBC, HGB, NEUT, MONO at each time point in each group are shown in FIG. 2. As disease progression, continuous decrease of peripheral RBC number, HGB level and NEUT percent was observed in NOG-EXL:CBMC transplanted mice, with no significant changes in PLT number and MONO percent. After IMH001 treatment, the recovery of RBC and NEUT decrease was observed, especially for RBC with significantly higher cell number and HGB level compared with untreated at Day 56 (6.45 vs 8. 17, p<0.001). There was also a monocyte percent decrease in IMH001 treatment group, which was considered as the pharmacological effect of IMH001.

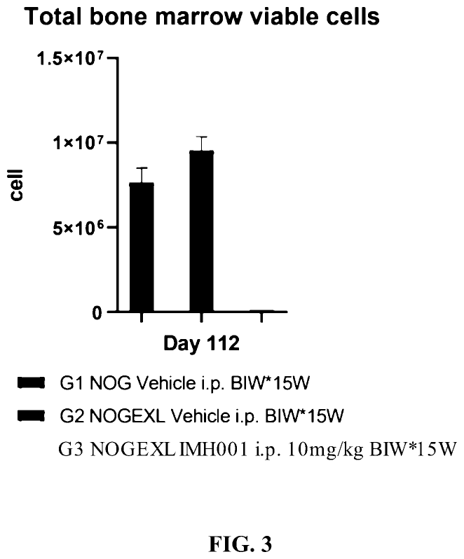

The total bone marrow viable cells are shown in FIG. 3. Compared with NOG-EXL untreated mice, there was an increase trend in total bone marrow cells at day 112 (mean value 9.55 vs 11.6, p=0.182). The number of peripheral RBC in MAS control group continued to decrease. Treatment of IMH001 could effectively improve RBC number in peripheral blood, accompanied by the HGB increase.

### 1.2.3 Ferritin, Fibrinogen and Triglyceride

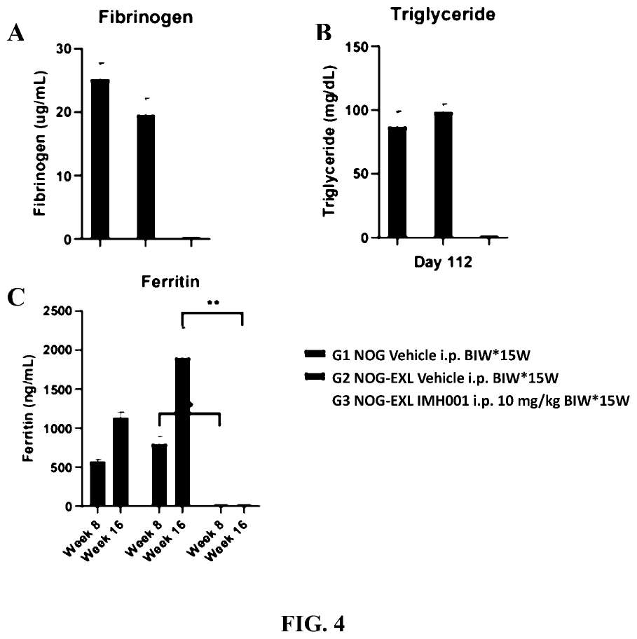

Serum was collected from mice at week 8 and 16. The ferritin, fibrinogen and triglycerides at each time point in each group are shown in FIG. 4. Ferritin, one indicator of macrophage activation, increase markedly as the disease progress in NOG-EXL CBMC transplanted mice, which was not observed in NOG CBMC transplanted mice. Moreover, compared with untreated, the IMH001 treatment can effectively control Ferritin level, which was observed at wk 8 and wk 16. No significant changes in Fibrinogen and Triglyceride were observed during the disease progress and after the IMH001 treatment.

The result showed that ferritin in peripheral blood of the MAS control group increased significantly, suggesting activation of macrophages. Treatment of IMH001 could significantly reduce the level of ferritin in peripheral blood.

1.2.4 Histo-Pathological evaluation

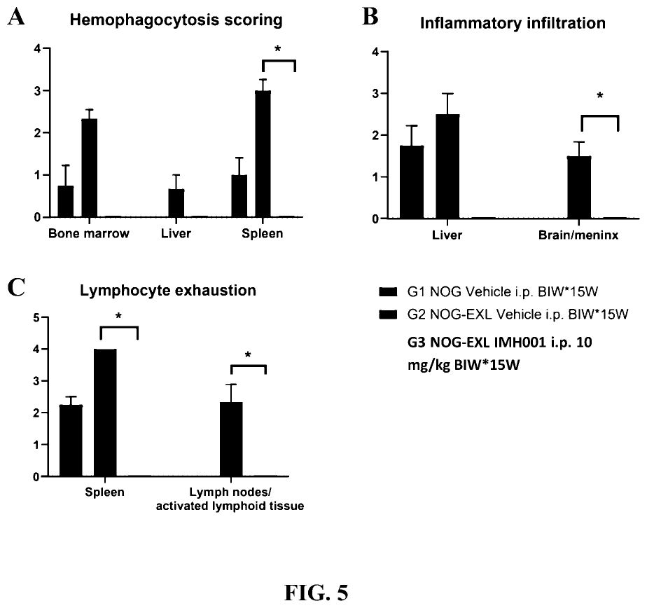

At study end (wk 16), cell smears from bone marrow, sections of liver and spleen were collected for Wright Giemsa staining to detect the presence of hemophagocytosis. Activated lymphoid tissue/lymph nodes and spleen were collected for H&E stained for lymphocyte exhaustion evaluation; liver and brain/meninges were H&E stained to detect inflammatory cell infiltration. The results are shown in FIG. 5.

Hemophagocytosis: Hemosiderin-containing macrophages were seen in spleen red pulp sections and bone marrow smears of the MAS control group, which indicates hemophagocytosis. Treatment of IMH001 could effectively prevent MAS related hemophagocytosis.

Lymphocyte exhaustion: The number of lymphoid follicles and germinal centers in the spleen and the number of lymphocytes in the lymph nodes decreased in NOG-EXL mice, which indicates lymphocyte exhaustion. Treatment of IMH001 could effectively reduce the lymphocyte exhaustion. Also, decreased inflammatory infiltration was observed in liver and meninx after IMH001 treatment.

The result showed that 16 weeks after CBMC transplantation, compared with NOG mice, NOG-EXL mice showed increased hemophagocytosis, elevated lymphocyte exhaustion and inflammatory. The above histopathology parameters were improved significantly in IMH001 treated mice.

Hemophagocytosis: Hemosiderin-containing macrophages were seen in spleen red pulp sections and bone marrow smears of the MAS control group, which indicates hemophagocytosis.

Lymphocyte exhaustion: The number of lymphoid follicles and germinal centers in the spleen and the number of lymphocytes in the lymph nodes decreased in NOG-EXL mice, which indicates lymphocyte exhaustion.

The study indicated that the treatment of IMH001 showed efficacy on all above parameters to delay disease progression as compared to tocilizumab.

### Example 2. Development of IMH001 Formulation

This example developed a suitable formulation for clinical use of IMH001 in treating MAS.

Three different buffer systems, sodium acetate, histidine hydrochloride and sodium dihydrogen phosphate, were initially evaluated, at various different pH levels, for IMH001. All three buffer systems were able to keep the antibody stable, and pH 5.5 to 6.0 appeared to be more beneficial than 6.5 or higher. Histidine hydrochloride and pH 5.8 was selected for further testing.

The next step evaluated a number of excipients, including sucrose, trehalose, mannitol, proline, arginine hydrochloride, glycine, solidum chloride, PS20 and PS80. According to the changes in stability (CEX-HPLC) results at 50° C. for 1 week, comparison was carried out for the formulation with different excipients. Results show that sucrose and proline were similar in performance and slightly better than the other formulae. Sucrose was selected for further development.

It was unknown, however, whether the selected buffer and excipient would allow a protein solubility of IMH001 suitable for subcutaneous injection at a high concentration. The solubility and viscosity of the protein at concentrations of 100 mg/ml and 150 mg/ml in histidine hydrochloride buffer system (pH 5.8) were therefore assessed.

In 20 mmol/L histidine hydrochloride buffer system (pH 5.8), when the protein was at concentrations of 100 mg/ml and 150 mg/ml and the samples were placed at 4° C. and 25° C. for 48 hours, no obvious changes were found in appearance color, clarity, OD350, protein concentration and viscosity, wherein the viscosity was lower than 10 cP and the clarity was lower than 6 NTU.

PS80 (Polysorbate 80) was then tested for its ability to improve durability and stability of the candidate formulations, at different concentrations of sucrose for a protein concentration of 100 mg/ml. The viscosity of all of the samples was acceptable, and the viscosity decreased with the decrease of sucrose concentration.

Upon further adjustment of each ingredient's concentration, the following formulation was determined to be superior to other candidate formulations: 100 mg/ml of IMH001, 20 mM histidine, 220 mM sucrose, and 0.02% (w/v) polysorbate 80, at pH 5.8. This formulation was used as stock solution for the following in vivo studies.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating or preventing macrophage activation syndrome (MAS) or hemophagocytic lymphohistiocytosis (HLH) in a patient in need thereof, comprising administering to the patient an antibody or fragment thereof having specificity to a human GM-CSF protein.

2. The method of claim 1, wherein the antibody comprises a heavy chain variable region (VH) comprising the amino acid sequence of SEQ ID NO:1 and a light chain variable region (VL) comprising the amino sequence of SEQ ID NO:2.

3. The method of claim 2, wherein the antibody comprises a human IgG1 Fc.

4. The method of claim 1, wherein the patient has a disease or condition selected from the group consisting of systemic-onset juvenile idiopathic arthritis (SoJIA), systemic lupus erythematosus (SLE), Kawasaki disease, and adult-onset Still's disease.

5. The method of claim 1, wherein the patient has a blood ferritin level that is at least 300 ng/ml, or at least 350 ng/ml, 400 ng/ml, 450 ng/ml, 500 ng/ml, 550 ng/ml, 600 ng/ml, 650 ng/ml, 684 ng/ml, 700 ng/ml, 720 ng/ml, 740 ng/ml, 750 ng/ml, 760 ng/ml, 784 ng/ml, 800 ng/ml, 820 ng/ml, 840 ng/ml, 850 ng/ml, 860 ng/ml, 884 ng/ml, 900 ng/ml, 950 ng/ml, or 1000 ng/ml.

6. The method of claim 1, wherein the patient has a hemoglobin level that is lower than 11 g/dL, or lower than 10.5 g/dL, 10 g/dL, 9.5 g/dL, 9 g/dL, 8.5 g/dL, 8 g/dL, 7.5 g/dL, or 7 g/dL.

7. The method of claim 1, wherein the patient has a platelet count that is lower than 140×109/L, or lower than 130×109/L, 120×109/L, 110×109/L, 100×109/L, 90×109/L, 80×109/L, 70×109/L, or60×109/L.

8. The method of claim 1, wherein the patient has a neutrophil count that is lower than 1.4×109/L, or lower than 1.3×109/L, 1.2×109/L, 1.1×109/L, 1.0×109/L, 0.9×109/L, 0.8×109/L, 0.7×109/L, or 0.6×109/L.

9. The method of claim 1, wherein the patient has a fasting triglyceride level that is higher than 2.49 mmol/L, or higher than 2.55 mmol/L, 2.6 mmol/L, 2.66 mmol/L, 2.72 mmol/L, 2.77 mmol/L, 2.83 mmol/L, 2.89 mmol/L, 2.94 mmol/L, 3 mmol/L, 3.06 mmol/L, 3.11 mmol/L, 3.17 mmol/L, 3.23 mmol/L, 3.28 mmol/L, 3.34 mmol/L, or 3.4 mmol/L.

10. The method of claim 1, wherein the patient has a blood fibrinogen level that is lower than 1.5 g/L, or lower than 1.4 g/L, 1.3 g/L, 1.2 g/L, 1.1 g/L, 1.0 g/L, 0.9 g/L, 0.8 g/L, 0.7 g/L, 0.6 g/L, or 0.5 g/L.

11. The method of claim 1, wherein the patient has an elevated soluble CD163 level as compared to a healthy individual.

12. The method of claim 1, wherein the patient has an elevated soluble IL-2 receptor level as compared to a healthy individual.

13. The method of claim 1, wherein the antibody or antigen binding fragment thereof is administered at 0.3 mg/kg to 25 mg/kg.

14. The method of claim 1, wherein the antibody or antigen binding fragment thereof is administered at 5 mg/kg to 20 mg/kg.

15. (canceled)

16. The method of claim 1, wherein the antibody or antigen binding fragment thereof is administered at 6 mg/kg or 10 mg/kg.

17. (canceled)

18. (canceled)

19. The method of claim 1, wherein the antibody is provided in a formulation comprising:
(a) 20 mg/ml-200 mg/ml of the antibody;
(b) 10 mM-30 mM of a buffering agent;
(c) 130 mM-250 mM of an isotonicity modifier; and
(d) 0.01% (w/v)-0.03% (w/v) of a surfactant,
wherein the formulation has a pH of 4.5-7.5.

20. The method of claim 19, wherein the formulation comprises 50-150 mg/ml of the antibody, 10-20 mM histidine, 200-220 mM sucrose, and 0.01-0.03% (w/v) polysorbate 80, at a pH of about 5.5-6.1.

21. The method of claim 20, wherein the formulation comprises about 100 mg/ml of the antibody, about 20 mM histidine, about 220 mM sucrose, and about 0.02% (w/v) polysorbate 80, at a pH of about 5.8.

22. The method of claim 1, further comprising administering to the patient a glucocorticoid, cyclosporine or anakinra.

23. The method of claim 22, wherein the glucocorticoid is selected from the group consisting of cortisol, cortisone, prednisone, prednisolone, methylprednisolone, dexamethasone, betamethasone, triamcinolone, deflazacort, fludrocortisone acetate, deoxycorticosterone acetate, aldosterone, and beclometasone.


SEQUENCE LISTING (2 sequences)

SEQ ID NO: 1 - protein, 120 aa, synthetic construct
EVQLVQSGAEVKKPGASVKVSCKASGYTFTSHYLHWVRQAPGQGLEWMGWIFPGDDKTKYNEKFKGRVTMTSDTSISTAYMELSRLRSDDTAVYYCARGTKYLNWNFDVWGQGTTVTVSS

SEQ ID NO: 2 - protein, 107 aa, synthetic construct
DIQLTQSPSFLSASVGDRVTITCKANQNVGTTLAWYQQKPGKSPKALIYSASYRYSGVPDRFSGSGSGTDFTLTISSLQPEDFATYFCHQYTTYPLTFGGGTKVEIK